(19) United States
(12) Patent Application Publication
(10) Pub. No.: US 2018/0049801 A1
(43) Pub. Date: Feb. 22, 2018

(54) METHODS FOR FACILITATION OF HEAT TRANSFER TO A VESSEL WALL IN HEAT INDUCED EMBOLIZATION

(71) Applicant: Cook Medical Technologies LLC, Bloomington, IN (US)

(72) Inventors: Rune T. Paamand, Broenshoej (DK); Per Elgaard, Haslev (DK)

(21) Appl. No.: 15/679,225

(22) Filed: Aug. 17, 2017

Related U.S. Application Data
(60) Provisional application No. 62/377,514, filed on Aug. 19, 2016.

(51) Int. Cl. A61B 18/14; A61N 1/05

(52) U.S. Cl. CPC A61B 18/1492; A61N 1/05

(57) ABSTRACT

The present disclosure provides for a device and methods of use to endoluminally heat and/or occlude a body vessel. The method includes a step of contracting the vessel to be treated by a contraction method, such as by delivering an electrical stimulus to induce spasms and decrease the diameter of the vessel. In some embodiments, the electrical stimulus may be a pulsatile electrical stimulus. The device then delivers a heat energy to aid in occlusion of the vessel.

### FIELD

The present disclosure relates generally to medical devices. More specifically, the disclosure relates to a device and method(s) for occluding or closing a body vessel using an electrical pulse to contract the body vessel in conjunction with using energy such as heat energy from an energy source to heat and/or emoblize the body vessel.

### BACKGROUND

There are numerous medical conditions when it is desired or necessary to close a body vessel, including the treatment of aneurysms, arteriovenous malformations, arteriovenous fistulas, for starving organs of oxygen and nutrients, in the treatment or containment of cancerous growths, and so on.

Several techniques are known and in use for closing or occluding such body vessels. Traditionally, vessels have been closed by means of external ligation, which generally must be carried out by an open surgery procedure, with its associated risks, inconvenience, and long patient recovery times. Other, more recent, methods aim to use an endoluminal procedure to insert into the vessel or organ one or more occlusion devices, such as a metal framed occluder, coils, pellets or the like, able to obstruct the flow of blood in the vessel.

In some cases, known devices are less suitable for certain types of endoluminal ablation procedures. For example, many proposed devices involve providing a catheter around which an electrically-conductive element is positioned. Such devices may be of too large a diameter to infiltrate vessels that require treatment, particularly small arteries. In some cases, particularly when the size of the vessel to be treated has a diameter that is significantly larger than the heating element, energy has a longer way to travel in order to affect heating of the vessel wall, and dissipates as it radiates away from the thermal tip. Moreover, blood, being a fluid that is in motion, acts as a natural heat sink, carrying thermal energy downstream from the site of heating and away from the site to be treated.

It has been a challenge to develop a small-diameter, flexible device which is robust enough to support an electrically-conductive element for an endoluminal heating device, as well as methods that efficiently allow for the use of a single heating element to treat vessels of varying sizes.

### BRIEF SUMMARY

The invention may include any of the following embodiments in various combinations and may also include any other aspect described below in the written description or in the attached drawings. This disclosure provides a medical device and methods for conducting vessel contraction, heating, and occlusion.

In one embodiment, a method of heating a treatment site of a body vessel in a body is described. The method includes positioning a device in the body vessel, the device having a transmission portion positioned adjacent the treatment site; delivering an electrical stimulus to the body vessel, the electrical stimulus having a frequency and an amplitude effective to cause vasoconstriction of the body vessel at the treatment site; and transmitting a heating energy from the transmission portion of the device to heat the body vessel. In some embodiments, the electrical stimulus may be a pulsatile electrical stimulus. The device may, in certain embodiments, provide the stimulatory or contraction electrical stimulus (in some cases, the pulsatile stimulus) intravascularly.

In another embodiment, a method of heating a treatment site of a body vessel is described. The method includes delivering an electrical stimulus to the body vessel, the electrical stimulus having a frequency and an amplitude effective to cause vasoconstriction of the body vessel at the treatment site; and transmitting a heating energy to the body vessel to heat the body vessel for occlusion. In some embodiments, the electrical stimulus may be a pulsatile electrical stimulus.

In a further embodiment, a system for heating a treatment site of a body vessel is described. The system includes a device comprising a transmission portion, the transmission portion comprising an electrically conductive element for positioning adjacent the treatment site, the transmission portion being capable of transmitting a heating energy to heat the body vessel; and a power source for delivering an electrical stimulus to the body vessel through the transmission portion, the electrical stimulus having a frequency and an amplitude effective to cause vasoconstriction of the body vessel at the treatment site; the transmission portion being in electrical connection with the power source. In some embodiments, the electrical stimulus may be a pulsatile electrical stimulus.

Various additional features and embodiments will become apparent with the following description. The present disclosure may be better understood by referencing the accompanying figures.

### BRIEF DESCRIPTION OF THE DRAWINGS

FIG. 1 depicts a side view of an endoluminal heating device in a blood vessel, in accordance with the principles of the present disclosure;

FIG. 2A depicts a side view of another embodiment of an endoluminal heating device in a blood vessel, in accordance with the principles of the present disclosure;

FIG. 2B is a close-up view of another embodiment of a monopolar device in accordance with the principles of the present disclosure;

FIG. 3 depicts a side view of a series of steps of sealing a vessel using a device and a method as described in the present disclosure; and

FIG. 4 is a flow chart documenting various steps of a method of sealing a vessel in accordance with the principles of the present disclosure.

### DETAILED DESCRIPTION

The present disclosure will now be described more fully with reference to the accompanying figures, which show various embodiments. The accompanying figures are provided for general understanding of various embodiments and method steps. However, this disclosure may be embodied in many different forms. These figures should not be construed as limiting, and they are not necessarily to scale. The following definitions will be used in this application.

“About” or “substantially” mean that two given quantities (e.g. lengths or volumes) are within 10%, preferably within 5%, more preferably within 1%. For example, a first quantity of length can be within 10% of a second length quantity.

“Adjacent” referred to herein is near, near to, or in close proximity with.

“Longitudinally” and derivatives thereof will be understood to mean along the longitudinal axis of the device.

“Radially” and derivatives thereof will be understood to mean along a radial axis of the body vessel.

The terms “proximal” and “distal” and derivatives thereof will be understood in the frame of reference of a physician using the device. Thus, proximal refers to locations closer to the physician and distal refers to the locations farther away from the physician (e.g., deeper in the patient's vasculature).

The present disclosure discusses a device 10 to perform vessel ablation by transmitting energy to a body vessel, particularly those having a blood flow therethrough. The target vessels for the present disclosure may be any vessel. For example, the target vessels may be small arteries, even those of about 1 millimeter or below 1 millimeter in diameter, that may be advantageously treated by a device with a low profile. It is envisioned that arteries in particular will be desirable target vessels, since these have strong enough walls where external compression is inadvisable (versus, for instance, veins.) In particular, the arteries of the liver and the kidney may be attractive for these procedures, as electrical stimulation in larger arteries near muscles may lead to unpleasant muscle contractions.

In this disclosure, a method for more efficiently sealing or occluding a body vessel, in particular a blood vessel, is described. The method involves delivering a signal to the vessel intraluminally, in some embodiments in the form of an electrical signal, in order to cause the vessel to contract. This allows for greater efficiency of heating and sealing, not only by causing the heating element of a device to be placed in closer proximity to the vessel wall itself, but in reducing the quantity of blood flowing past the treatment site and acting as a heat sink. This in turn leads to shorter and ostensibly less arduous procedures. Moreover, with the proper construction, a single device can be used for both delivery of the stimulating signal and for heating.

The device, in operation, may give rise to blood clotting, heating the blood surrounding the electrical element, and/or to inflame the body vessel to close the vessel. This can be achieved by selecting an energy level and/or time duration suitable to heat surrounding blood or tissue, which in some circumstances can be expected to include ablation of the vessel tunica, whereby there may be experienced some contraction of the vessel as a result of the heating. The skilled person will be able to determine suitable parameters from common general knowledge in the art. It is to be appreciated that parameters of the application will be dependent upon factors including the size of the vessel, the amount of blood flow through the vessel, pulsation and turbulence of blood at the treatment site, and so on. During a procedure or occlusion of a blood vessel, the flow rate of the blood flowing through the vessel will decrease as the blood vessel becomes constricted. As the blood flow decreases, the cooling aspects of the flowing blood will also decrease, leading to a resultant increase in efficiency of heating.

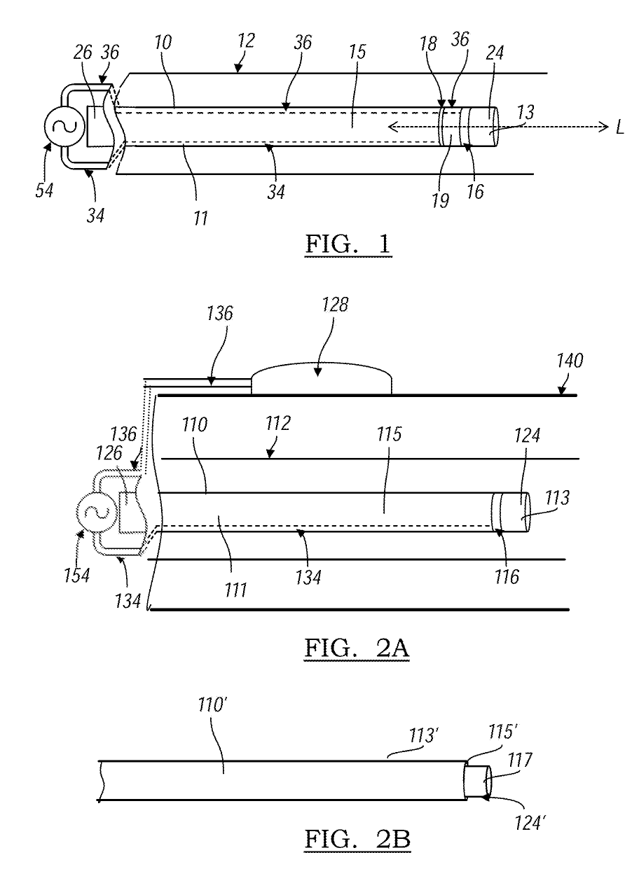

FIG. 1 depicts a side view of one embodiment of a medical device 10 for use in a system that may be used to heat a body vessel at or adjacent a treatment site. The device may have a support 11, which may be an elongate, substantially cylindrical member, or a mandrel, that extends from a proximal end 26 to a distal end 24. The support 11 may define a longitudinal axis. The device may have a curvature or be capable of being curved or bent similar to a common wire guide. In some embodiments, the support 11 may be a wire guide. The support 11, having wire guide characteristics, allows for flexibility between the proximal and distal ends of the device 10, which assists in navigation of tortuous vessels. The distal end 24 of the support 11 may be an atraumatic end. This atraumatic end may be rounded off in shape, or may be covered, for instance with a pliable or insulating layer. Such an atraumatic end may increase safety as the device is used.

The support 11 may have an outer diameter that distally decreases to form a distal taper. Alternatively, the support 11, as shown in FIG. 1, may have an outer diameter being uniform from the proximal end 26 to the distal end 24 (that is, substantially the same radius around longitudinal axis L). A distal taper, or a tapered tip, may be effective to minimize the overall diameter of the device and may make the distal end 24 more flexible and easier to track within the body vessel.

The support 11 itself may be substantially any type of elongate member that is suitable for intravascular procedures, such as a wire guide or a catheter. If a catheter, the support 11 will not necessarily have a lumen running therethrough, but may instead be a generally solid length of insulative polymer.

Generally, the device 10 of FIG. 1 may have a transmission portion that contains two conductive elements 16 and 18, which are operable to create an electric field. As both conductive elements 16 and 18 are located within vessel 12, when the device is in use, the device of FIG. 1 is an example of a bipolar device. In a bipolar system, electrical energy applied to the first electrode will pass by conduction to the second electrode. There can be localized heating at the electrodes, which affects the desired vessel heating and sealing. In one example, applying a radiofrequency (“RF”) signal generates the field, and the field heats the local environment in the body vessel (including the blood flow and the vessel wall) to cause swelling and occluding of the body vessel at a target site.

Conductive elements 16 and 18 may be formed as rings surrounding the exterior 15 of the support 11, with electrical connections to wires 34/36. In another embodiment, the conductive elements 16 and 18 may be portions of a wire guide that serves as the support 11, with metallic portions exposed through an insulating layer that has been removed. The conductive elements 16 and 18 may be of a largely symmetrical design (that is, they are substantially the same size) or they may be different sizes. In certain embodiments, the conductive elements may be spaced as little as 1 millimeter apart, or 0.5 millimeters apart, or even 0.25 millimeters apart.

The conductive elements may operate in a number of modes corresponding to the operation of the system for occlusion. For instance, the device 10 may operate to deliver both a stimulating electrical signal, such as in certain embodiments a pulsatile signal, in order to stimulate contraction of the vessel, and then in a heating mode (such as a substantially continuous mode) to provide heat energy to affect sealing.

When the device 10 operates, in general, stimulation to affect contraction of the target vessel is achieved via transmission of a relatively lower-frequency signal, typically about 10 kHz, as compared to an RF heating signal that is transmitted with a frequency of about 100 kilohertz (kHz) to about 1000 kHz, preferably between about 200 kHz and 500 kHz, preferably about 500 kHz. The average power of the stimulatory transmission may be relatively small, such as below about 1 watt, whereas the power of the heating is relatively high, at least about 5 watts. The average power of both signals (the stimulation signal and the RF current) may be adjusted in any way as is known in the art, such as by adjusting amplitude, adjusting applied voltage, and pulsing.

In one embodiment, the stimulatory signal may be a direct current at a relatively low power (about 1 watt or less). In one embodiment, such a direct current may be delivered as a series of pulses of short duration (in the microsecond range.)

In one embodiment, the stimulatory signal may be a charge balanced electrical signal, such that the sum of the signal (positive and negative) is zero within a period of time.

In one embodiment, the stimulatory signal may be a pulsatile electrical signal, and the heating signal may be a continuous RF signal.

In another embodiment, the stimulatory signal may have an amplitude of about 10 volts (V) to about 150 V, preferably about 80 V.

In another embodiment, the stimulatory signal may be a biphasic squarewave with a pulse width of about 10 microseconds to about 1000 microseconds, or about 10 microseconds to about 100 microseconds, preferably about 10 microseconds.

In still another embodiment, the heating signal may be a pulsatile amplitude modulated sinusoidal signal.

In another embodiment, the stimulatory signal may be pulsed with repetition rates between about 100 hertz (Hz) to about 100 kHz, or about 1 kHz to about 10 kHz.

In another embodiment, the stimulatory signal and the heating signal may operate simultaneously. In another specific embodiment, the stimulatory signal may be sufficient to affect contraction of a vessel, and may also generate sufficient heat to also effectively seal the vessel.

Returning to FIG. 1, the device 10 may further have a first insulated wire 34 (used interchangeably with the term “first wire”) and a second insulated wire 36 (or second wire) for transferring electrical current to and from the conductive elements 16 and 18 of the transmission portion of the device 10. The first wire 34 may be electrically coupled or connected to a power source 54 or signal generator 54 and may extend through the interior 15 of the support 11 to first conductive element 18. (Dotted lines represent wire running through the interior 15 of the device.) Likewise, the second wire 36 may be electrically coupled or connected to the power source 54 or signal generator 54 and may extend through the interior 15 of the support 11 to second conductive element 16. A space 19 may exist between the first and second conductive elements 16 and 18.

Although it is preferred to have the wires 34/36 run through the interior 15 of the support 11 to keep the overall profile of device 10 low, it is also possible to have the wires 34/36 adjoined to the exterior 13 of the device instead.

In general, the first and second insulated wires 34 and 36 will be standard electrical wires, comprising an insulating outer jacket or outer layer with a high dielectric constant that prohibits transmission of electricity therethrough under normal operating conditions, such as an enameled varnish, shrink tubing, molding, or a polymer layer. An inner conductive wire which is surrounded by the outer jacket may be made of a highly conductive material, including but not limited to copper, silver, and platinum. Ways of achieving connection to the power source 54 can include, but are not limited to, soldering, laser welding, press fitting, and so forth.

The device 10 may optionally have shrink tubing or some other insulative means of confinement disposed about portions thereof. An exemplary shrink tubing may extend around the support 11, the first wire 34, the second wire 36, and/or any other portion of the device 10. The shrink tubing may immobilize or bind the support 11, the first wire 34, and the second wire 36 in place so that they are immobilized relative to each other. A shrink tubing or insulator may in some embodiments be disposed about the support 11, in which case it may act to isolate or insulate the support 24 from the rest of the device 10.

The power supply 54 may be any suitable source of electrical current such that the operating parameters of the device 10 can be met. In one embodiment, the power source may be a battery, a source of direct current, or a source of alternating current (AC). The power supply 54 may in some embodiments be a battery and may be coupled to an inverter for the generation of alternating current. In some embodiments, the power supply 54 is operable in multiple modes, such as a pulsatile mode for generating stimulatory pulses to cause contraction of vessels, and a heating mode where a substantially continuous flow of electricity is provided in order to heat and seal the vessel. In some embodiments, the frequencies or waveforms of the signals in the stimulatory (contraction) mode and the heating mode differ. In some embodiments, the stimulatory and the heating modes may be active simultaneously, which may be possible due to this difference in frequencies or waveforms. In certain embodiments, the power supply 54 may include two different generating portions for the generation of each signal (stimulatory and heating). In another embodiment, a single generating portion may generate both signals at two frequencies.

In another embodiment, a monopolar device 110 is depicted in FIG. 2A. A monopolar system may have an elongate support 111 bearing a first electrode 116 (e.g. active electrode) and a second electrode 128 (e.g. dispersive electrode) pad or return electrode positioned outside the patient's body 140. The first electrode is designed to be fed endoluminally into the patient's vessel 112, while the second electrode pad 128 is positioned against the person's outer body, either close to or remote from the first electrode 116. In some embodiments, the pad 128 may be significantly larger than the first electrode 116, such as having at least three to four times the surface area of the first electrode 116, and in some cases being much larger. For example, in one embodiment, the first electrode 116 may have an overall surface area of about 1 square centimeter (cm²) or smaller, preferably 0.2 cm², and the second electrode 128 is a pad of about 10 cm in width and 10 cm in length, for a surface area of about 100 cm². The pad may be as large as is practical, even larger than 100 cm².

FIG. 2B depicts a different embodiment of a device 110′ of a monopolar design. In this embodiment, the support 111′ is a wire guide with an outer polymer coat and an inner conductive metal core. At distal end 124′, a portion of the polymer coat has been stripped away to expose tip 117′, which functions as a first electrode. Thus the design can be simplified and the entire wire guide 111′ electrified in order to generate the signal.

The other components described in conjunction with the disclosure of the bipolar device (e.g., the power source, the insulated wires, the overall structure of the support, and so forth) also apply to the device of monopolar construction.

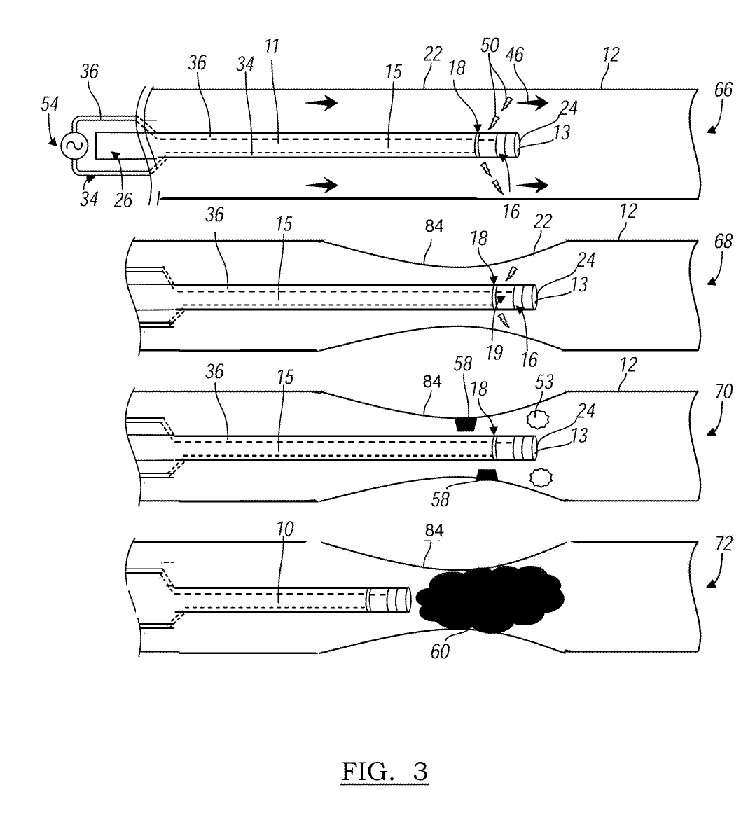

FIG. 3 depicts a method to fully occlude a body vessel 12. In first step 66, the device may be positioned in the body vessel 12 adjacent the treatment site 22. At this time, blood flow 46 will be substantially normal and flowing at a first rate. As depicted, the system may generate a stimulatory electrical signal 50 from the power supply 54 and through one of first and second conductive elements 16/18, or both conductive elements. In one embodiment, the stimulatory signal is a pulsatile signal, which causes bursts of current to flow through the vessel and stimulate the nerves or muscle to initiate contraction of the vasculature.

In a second step 68, the body vessel 12 has begun to contract to form a contracted portion 84 at treatment site 22. Therefore, the power supply 54 has been switched from a first stimulatory mode of providing energy, to a second heating mode. In one embodiment, the first mode may be a pulsatile stimulatory mode, and the second mode may be a substantially continuous heating mode. If both conductive elements were active, switching modes may cause one to remain active, and the other to become passive, or act as a return electrode in order to complete the circuit.

This step of heating 70 with thermal energy 53 may cause swelling of the body vessel 12 at the treatment site, along with charring 58 of the blood, of the vessel wall, or both. As shown, the step 70 of transmitting thermal energy 53 may involve avoiding contact of the device 10 with the vessel wall. However, in some instances it is also possible that contact between the vessel wall and the heating device 10 may prove advantageous, and providing direct contact between the device 10 and a portion of the body vessel, such as an inner wall, will allow to heat the wall more efficiently and cause it to swell.

In step 72, the device 10 has generated sufficient current with power supply 54, and the vessel 12 has swollen and been heated to the point that occlusive closure 60 has formed. At this point, the step of transmitting a current has stopped as full occlusion has been achieved. As such, the operator may withdraw the device 10 from the body vessel 12. This withdrawal may be manual or automatic.

The operator may detect the occlusion process by assessing the temperature of the device and electrical impedance within the body vessel. Occlusion may be detected as an increase in temperature without an increase in output power, or in more general terms as a change in the power-to-temperature response, signifying a change in environment or cooling of the device as a result of occlusion. The progress of occlusion may also be monitored visually, via fluoroscopy or X-ray viewing. However, in many cases the vessel closure will simply be done by a timing method and by using predetermined electrical parameters, as visual inspection will prove difficult.

Such parameters may include predetermination of the characteristics of the stimulatory, pulsatile signal. In this case, a stimulatory signal may be a biphasic square wave signal with a pulse width of about 10 microseconds to about 1000 microseconds, or about 10 microseconds, per phase. The pulse may have an amplitude of about 10 volts to 150 volts, or about 80 volts. The pulses may have a repetition frequency of about 100 Hz to about 100 kHz, or about 1 kHz to about 10 kHz, or about 10 kHz. The overall duration of the pulsatile current exposure would be from about 2 seconds to about 120 seconds, or about 10 seconds to about 60 seconds

In the case of the heating energy, a radiofrequency signal of between about 100 kilohertz and about 1000 kilohertz, or about 200 kilohertz to about 500 kilohertz, may be provided through the conductive element(s) as a continuous or amplitude modulated sine wave.

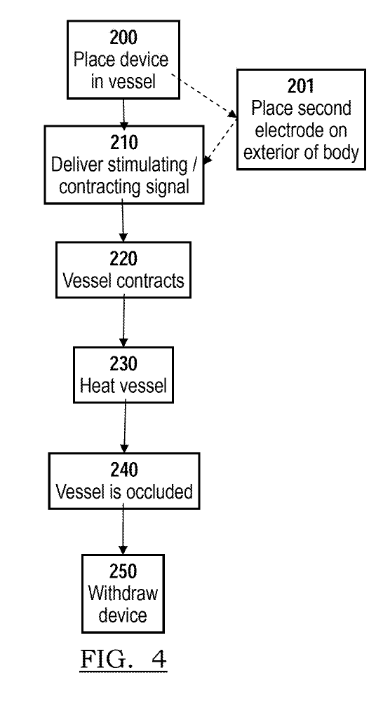

FIG. 4 provides a flow chart for the sequence of these steps. It is noted that the step of constriction of the vessel by pulsatile flow may precede heating, or may be carried out simultaneously. In a first step 200, the device (bearing at least one conductive element or electrode) is placed in the vessel. In the case of a monopolar device, step 201, wherein a second electrode is placed on the body, is conducted. Then a pulsatile signal generated from the power supply or generator is delivered in step 210. In step 220, the vessel contracts due to the pulsatile signal. While contraction is occurring, or after it has occurred, the vessel is heated in step 230. Heat is supplied until the vessel is occluded in step 240. Finally, in step 250, the device is withdrawn.

In some embodiments, heating intraluminally is not restricted to the use of a radiofrequency signal. Heat may be supplied by a laser or other optical device, by a resistive heating mechanism, or any other suitable method. It will be appreciated that different heating strategies will give rise to different equipment needs (e.g., laser heating will involve incorporating a laser source onto the support for intraluminal delivery.)

It should be understood that the foregoing relates to exemplary embodiments of the disclosure and that modifications may be made without departing from the spirit and scope of the disclosure as set forth in the following claims. While the disclosure has been described with respect to certain embodiments it will be appreciated that modifications and changes may be made by those skilled in the art without departing from the spirit of the disclosure.

1. A method of heating a treatment site of a body vessel in a body, the method comprising:
positioning a device in the body vessel, the device having a transmission portion positioned adjacent the treatment site;
delivering an electrical stimulus to the body vessel, the electrical stimulus having a frequency and an amplitude effective to cause vasoconstriction of the body vessel at the treatment site; and
transmitting a heating energy from the transmission portion of the device to heat the body vessel.

2. The method of claim 1, wherein the device provides a pulsatile electrical stimulus intravascularly.

3. The method of claim 1, wherein the heating energy comprises a radiofrequency signal.

4. The method of claim 3, wherein the radiofrequency signal has a frequency from about 100 kilohertz to about 1000 kilohertz.

5. The method of claim 4, wherein the frequency of the radiofrequency signal is about 500 kilohertz.

6. The method of claim 1, wherein the device comprises a bipolar device.

7. The method of claim 1, wherein the device comprises a monopolar device.

8. The method of claim 1, wherein the electrical stimulus comprises a pulsatile electrical stimulus having a repetition frequency from about 100 hertz to about 100 kilohertz.

9. The method of claim 1, wherein each pulse of the pulsatile electrical stimulus has duration from about 10 microsecond to about 1000 microseconds per phase.

10. The method of claim 1, wherein the pulsatile electrical stimulus has a square waveform.

11. The method of claim 1, wherein the pulsatile electrical stimulus is provided for about 2 seconds to about 120 seconds.

12. The method of claim 1, wherein the step of delivering the pulsatile electrical stimulus is conducted prior to transmitting the heating energy.

13. The method of claim 1, wherein the steps of delivering the electrical stimulus and transmitting the heating energy are simultaneous.

14. The method of claim 1, wherein the method comprises constricting the body vessel such that a portion of the body vessel directly contacts the device.

15. The method of claim 1, wherein the electrical stimulus comprises a direct current.

16. The method of claim 1, wherein the heating energy is energy from at least one of a laser, an optical source, and a resistive heating element.

17. A method of heating a treatment site of a body vessel, the method comprising:
delivering an electrical stimulus to the body vessel, the electrical stimulus having a frequency and an amplitude effective to cause vasoconstriction of the body vessel at the treatment site; and
transmitting a heating energy to the body vessel to heat the body vessel for occlusion.

18. The method of claim 17, further comprising delivering the electrical stimulus to the body vessel by a bipolar device.

19. A system for heating a treatment site of a body vessel, the system comprising:
a device comprising a transmission portion, the transmission portion comprising an electrically conductive element for positioning adjacent the treatment site, the transmission portion being capable of transmitting a heating energy to heat the body vessel; and
a power source for delivering an electrical stimulus to the body vessel through the transmission portion, the electrical stimulus having a frequency and an amplitude effective to cause vasoconstriction of the body vessel at the treatment site;
the transmission portion being in electrical connection with the power source.

20. The system of claim 19, wherein the device comprises a monopolar device.

21. The system of claim 19, wherein the device comprises a bipolar device.

22. The system of claim 19, wherein the transmission portion comprises at least one of a laser, an optical source, and a resistive heating element.
